(19) United States
(12) Patent Application Publication
(10) Pub. No.: US 2006/0116541 A1
(43) Pub. Date: Jun. 1, 2006

(54) Oxygenate conversion using boron-containing molecular sieve CHA

(75) Inventors: Lun-Teh Yuen, San Francisco, CA (US); Stacey I. Zones, San Francisco, CA (US)

Correspondence Address: CHEVRON TEXACO CORPORATION, P.O. BOX 6006, SAN RAMON, CA, 94583-0806, US

(73) Assignee: Chevron U.S.A. Inc.

(21) Appl. No.: 11/266,084

(22) Filed: Nov. 2, 2005

Related U.S. Application Data
(60) Provisional application No. 60/632,007, filed on Nov. 30, 2004.

(51) Int. Cl. C07C 1/00; C07C 1/20

(52) U.S. Cl. 585/639; 585/640

(57) ABSTRACT

A boron-containing molecular sieve having the CHA crystal structure and comprising (1) silicon oxide and (2) boron oxide or a combination of boron oxide and aluminum oxide, iron oxide, titanium oxide, gallium oxide and mixtures thereof is prepared using a quaternary ammonium cation derived from 1-adamantamine, 3-quinuclidinol or 2-exo-aminonorbornane as structure directing agent. The molecular sieve can be used for gas separation or in catalysts to prepare methylamine or dimethylamine, to convert oxygenates (e.g., methanol) to light olefins, or for the reduction of oxides of nitrogen n a gas stream (e.g., automotive exhaust).

This application claims benefit under 35 USC 119 of Provisional Application 60/632007, filed Nov. 30, 2004.

### BACKGROUND

Chabazite, which has the crystal structure designated “CHA”, is a natural zeolite with the approximate formula Ca₆Al₁₂Si₂₄O₇₂. Synthetic forms of chabazite are described in “Zeolite Molecular Sieves” by D. W. Breck, published in 1973 by John Wiley & Sons. The synthetic forms reported by Breck are: zeolite “K-G”, described in J. Chem. Soc., p. 2822 (1956), Barrer et al.; zeolite D, described in British Patent No. 868,846 (1961); and zeolite R, described in U.S. Pat. No. 3,030,181, issued Apr. 17, 1962 to Milton et al. Chabazite is also discussed in “Atlas of Zeolite Structure Types” (1978) by W. H. Meier and D. H. Olson.

The K-G zeolite material reported in the J. Chem. Soc. Article by Barrer et al. is a potassium form having a silica:alumina mole ratio (referred to herein as “SAR”) of 2.3:1 to 4.15:1. Zeolite D reported in British Patent No. 868,846 is a sodium-potassium form having a SAR of 4.5:1 to 4.9:1. Zeolite R reported in U.S. Pat. No. 3,030,181 is a sodium form which has a SAR of 3.45:1 to 3.65:1.

Citation No. 93:66052y in Volume 93 (1980) of Chemical Abstracts concerns a Russian language article by Tsitsishrili et al. in Soobsch. Akad. Nauk. Gruz. SSR 1980, 97(3) 621-4. This article teaches that the presence of tetramethylammonium ions in a reaction mixture containing K₂O—Na₂O—SiO₂—Al₂O₃—H₂O promotes the crystallization of chabazite. The zeolite obtained by the crystallization procedure has a SAR of 4.23.

The molecular sieve designated SSZ-13, which has the CHA crystal structure, is disclosed in U.S. Pat. No. 4,544,538, issued Oct. 1, 1985 to Zones. SSZ-13 is prepared from nitrogen-containing cations derived from 1-adamantamine, 3-quinuclidinol and 2-exo-aminonorbornane. Zones discloses that the SSZ-13 of U.S. Pat. No. 4,544,538 has a composition, as-synthesized and in the anhydrous state, in terms of mole ratios of oxides as follows:

(0.5 to 1.4)R₂O : (0 to 0.5)M₂O : W₂O₃ : (greater than 5)YO₂ wherein M is an alkali metal cation, W is selected from aluminum, gallium and mixtures thereof, Y is selected from silicon, germanium and mixtures thereof, and R is an organic cation. U.S. Pat. No. 4,544,538 does not, however, disclose boron-containing SSZ-13.

U.S. Pat. No. 6,709,644, issued Mar. 23, 2004 to Zones et al., discloses zeolites having the CHA crystal structure and having small crystallite sizes. It does not, however, disclose a CHA zeolite containing boron. It is disclosed that the zeolite can be used for separation of gasses (e.g., separating carbon dioxide from natural gas), and in catalysts used for the reduction of oxides of nitrogen in a gas stream (e.g., automotive exhaust), converting lower alcohols and other oxygenated hydrocarbons to liquid products, and for producing dimethylamine.

### SUMMARY OF THE INVENTION

The present invention relates to a process for the production of light olefins comprising olefins having from 2 to 4 carbon atoms per molecule from an oxygenate feedstock. The process comprises passing the oxygenate feedstock to an oxygenate conversion zone containing a molecular sieve catalyst to produce a light olefin stream.

Thus, in accordance with the present invention there is provided a process for the production of light olefins from a feedstock comprising an oxygenate or mixture of oxygenates, the process comprising reacting the feedstock at effective conditions over a catalyst comprising boron-containing molecular sieve having the CHA crystal structure and comprising (1) silicon oxide and (2) boron oxide or a combination of boron oxide and aluminum oxide, iron oxide, titanium oxide, gallium oxide and mixtures thereof.

### DETAILED DESCRIPTION

The present invention relates to molecular sieves having the CHA crystal structure and containing boron in their crystal framework.

Boron-containing CHA molecular sieves can be suitably prepared from an aqueous reaction mixture containing sources of sources of an oxide of silicon; sources of boron oxide or a combination of boron oxide and aluminum oxide, iron oxide, titanium oxide, gallium oxide and mixtures thereof; optionally sources of an alkali metal or alkaline earth metal oxide; and a cation derived from 1-adamantamine, 3-quinuclidinol or 2-exo-aminonorbornane. The mixture should have a composition in terms of mole ratios falling within the ranges shown in Table A below:

TABLE A

| | |
|---|---|
| YO₂/WₐO_{b} | >2-2,000 |
| OH—/YO₂ | 0.2-0.45 |
| Q/YO₂ | 0.2-0.45 |
| M_{2/n}O/YO₂ | 0-0.25 |
| H₂O/YO₂ | 22-80 |

wherein Y is silicon,; W is boron or a combination of boron and aluminum, iron, titanium, gallium and mixtures thereof; M is an alkali metal or alkaline earth metal; n is the valence of M (i.e., 1 or 2) and Q is a quaternary ammonium cation derived from 1-adamantamine, 3-quinuclidinol or 2-exo-aminonorbornane (commonly known as a structure directing agent or “SDA”).

The quaternary ammonium cation derived from 1-adamantamine can be a N,N,N-trialkyl-1-adamantammonium cation which has the formula: 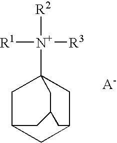
where R¹, R², and R³ are each independently a lower alkyl, for example methyl. The cation is associated with an anion, A⁻, which is not detrimental to the formation of the molecular sieve. Representative of such anions include halogens, such as fluoride, chloride, bromide and iodide; hydroxide; acetate; sulfate and carboxylate. Hydroxide is the preferred anion. It may be beneficial to ion exchange, for example, a halide for hydroxide ion, thereby reducing or eliminating the alkali metal or alkaline earth metal hydroxide required.

The quaternary ammonium cation derived from 3-quinuclidinol can have the formula: 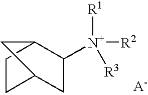
where R¹, R², R³ and A are as defined above.

The quaternary ammonium cation derived from 2-exo-aminonorbornane can have the formula: 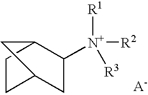
where R¹, R², R³ and A are as defined above.

The reaction mixture is prepared using standard molecular sieve preparation techniques. Typical sources of silicon oxide include fumed silica, silicates, silica hydrogel, silicic acid, colloidal silica, tetra-alkyl orthosilicates, and silica hydroxides. Sources of boron oxide include borosilicate glasses and other reactive boron compounds. These include borates, boric acid and borate esters. Typical sources of aluminum oxide include aluminates, alumina, hydrated aluminum hydroxides, and aluminum compounds such as AlCl₃ and Al₂(SO₄)₃. Sources of other oxides are analogous to those for silicon oxide, boron oxide and aluminum oxide.

It has been found that seeding the reaction mixture with CHA crystals both directs and accelerates the crystallization, as well as minimizing the formation of undesired contaminants. In order to produce pure phase boron-containing CHA crystals, seeding may be required. When seeds are used, they can be used in an amount that is about 2-3 weight percent based on the weight of YO₂.

The reaction mixture is maintained at an elevated temperature until CHA crystals are formed. The temperatures during the hydrothermal crystallization step are typically maintained from about 120° C. to about 160° C. It has been found that a temperature below 160° C., e.g., about 120° C. to about 140° C., is useful for producing boron-containing CHA crystals without the formation of secondary crystal phases.

The crystallization period is typically greater than 1 day and preferably from about 3 days to about 7 days. The hydrothermal crystallization is conducted under pressure and usually in an autoclave so that the reaction mixture is subject to autogenous pressure. The reaction mixture can be stirred, such as by rotating the reaction vessel, during crystallization.

Once the boron-containing CHA crystals have formed, the solid product is separated from the reaction mixture by standard mechanical separation techniques such as filtration. The crystals are water-washed and then dried, e.g., at 90° C. to 150° C. for from 8 to 24 hours, to obtain the as-synthesized crystals. The drying step can be performed at atmospheric or subatmospheric pressures.

The boron-containing CHA molecular sieve has a composition, as-synthesized and in the anhydrous state, in terms of mole ratios of oxides as indicated in Table B below:

### As-Synthesized Boron-Containing CHA Composition

TABLE B

| | |
|---|---|
| YO₂/W_{c}O_{d} | 20-2,000 |
| M_{2/n}O/YO₂ | 0-0.03 |
| Q/YO₂ | 0.02-0.05 |

where Y, W, M, n and Q are as defined above.

The boron-containing CHA molecular sieves, as-synthesized, have a crystalline structure whose X-ray powder diffraction (“XRD”) pattern shows the following characteristic lines:

TABLE I
As-Synthesized Boron-Containing CHA XRD

| 2 Theta^{(a)} | d-spacing (Angstroms) | Relative Intensity^{(b)} |
|---|---|---|
| 9.68 | 9.13 | S |
| 14.17 | 6.25 | M |
| 16.41 | 5.40 | VS |
| 17.94 | 4.94 | M |
| 21.13 | 4.20 | VS |
| 25.21 | 3.53 | VS |
| 26.61 | 3.35 | W-M |
| 31.11 | 2.87 | M |
| 31.42 | 2.84 | M |
| 31.59 | 2.83 | M |

^{(a)}±0.10
^{(b)}The X-ray patterns provided are based on a relative intensity scale in which the strongest line in the X-ray pattern is assigned a value of 100: W(weak) is less than 20; M(medium) is between 20 and 40; S(strong) is between 40 and 60; VS(very strong) is greater than 60.

Table IA below shows the X-ray powder diffraction lines for as-synthesized boron-containing CHA including actual relative intensities.

TABLE IA
As-Synthesized Boron-Containing CHA XRD

| 2 Theta^{(a)} | d-spacing (Angstroms) | Relative Intensity (%) |
|---|---|---|
| 9.68 | 9.13 | 55.2 |
| 13.21 | 6.70 | 5.4 |
| 14.17 | 6.25 | 33.5 |
| 16.41 | 5.40 | 81.3 |
| 17.94 | 4.94 | 32.6 |
| 19.43 | 4.56 | 6.8 |
| 21.13 | 4.20 | 100 |
| 22.35 | 3.97 | 15.8 |
| 23.00 | 3.86 | 10.1 |
| 23.57 | 3.77 | 5.1 |
| 25.21 | 3.53 | 78.4 |
| 26.61 | 3.35 | 20.2 |
| 28.37 | 3.14 | 6.0 |
| 28.57 | 3.12 | 4.4 |
| 30.27 | 2.95 | 3.9 |
| 31.11 | 2.87 | 29.8 |
| 31.42 | 2.84 | 38.3 |
| 31.59 | 2.83 | 26.5 |
| 32.27 | 2.77 | 1.4 |
| 33.15 | 2.70 | 3.0 |
| 33.93 | 2.64 | 4.7 |
| 35.44 | 2.53 | 3.9 |
| 35.84 | 2.50 | 1.2 |
| 36.55 | 2.46 | 10.9 |
| 39.40 | 2.29 | 1.8 |
| 40.02 | 2.25 | 1.3 |
| 40.44 | 2.23 | 1.0 |
| 40.73 | 2.21 | 6.0 |

^{(a)}±0.10

After calcination, the boron-containing CHA molecular sieves have a crystalline structure whose X-ray powder diffraction pattern include the characteristic lines shown in Table II:

TABLE II
Calcined Boron-Containing CHA XRD

| 2 Theta^{(a)} | d-spacing (Angstroms) | Relative Intensity |
|---|---|---|
| 9.74 | 9.07 | VS |
| 13.12 | 6.74 | M |
| 14.47 | 6.12 | W |
| 16.38 | 5.41 | W |
| 18.85 | 4.78 | M |
| 21.07 | 4.21 | M |
| 25.98 | 3.43 | W |
| 26.46 | 3.37 | W |
| 31.30 | 2.86 | W |
| 32.15 | 2.78 | W |

^{(a)}±0.10

Table IIA below shows the X-ray powder diffraction lines for calcined boron-containing CHA including actual relative intensities.

TABLE IIA
Calcined Boron-Containing CHA XRD

| 2 Theta^{(a)} | d-spacing (Angstroms) | Relative Intensity (%) |
|---|---|---|
| 9.74 | 9.07 | 100 |
| 13.12 | 6.74 | 29.5 |
| 14.47 | 6.12 | 4.6 |
| 16.38 | 5.41 | 14.2 |
| 18.85 | 4.78 | 22.1 |
| 19.60 | 4.53 | 2.2 |
| 21.07 | 4.21 | 32.9 |
| 22.84 | 3.89 | 2.2 |
| 23.68 | 3.75 | 0.8 |
| 25.98 | 3.43 | 13.1 |
| 26.46 | 3.37 | 8.7 |
| 28.27 | 3.15 | 1.3 |
| 29.24 | 3.05 | 1.6 |
| 30.32 | 2.95 | 1.7 |
| 31.30 | 2.86 | 14.4 |
| 32.15 | 2.78 | 9.0 |
| 32.56 | 2.75 | 0.2 |
| 35.26 | 2.54 | 2.4 |

^{(a)}±0.15

The X-ray powder diffraction patterns were determined by standard techniques. The radiation was the K-alpha/doublet of copper and a scintillation counter spectrometer with a strip-chart pen recorder was used. The peak heights I and the positions, as a function of 2 Theta where Theta is the Bragg angle, were read from the spectrometer chart. From these measured values, the relative intensities, 100×I/lo, where lo is the intensity of the strongest line or peak, and d, the interplanar spacing in Angstroms corresponding to the recorded lines, can be calculated.

Variations in the diffraction pattern can result from variations in the mole ratio of oxides from sample to sample. The molecular sieve produced by exchanging the metal or other cations present in the molecular sieve with various other cations yields a similar diffraction pattern, although there can be shifts in interplanar spacing as well as variations in relative intensity. Calcination can also cause shifts in the X-ray diffraction pattern. Also, the symmetry can change based on the relative amounts of boron and aluminum in the crystal structure. Notwithstanding these perturbations, the basic crystal lattice structure remains unchanged.

The present invention comprises a process for catalytic conversion of a feedstock comprising one or more oxygenates comprising alcohols and ethers to a hydrocarbon product containing light olefins, i.e., C₂, C₃ and/or C₄ olefins. The feedstock is contacted with the molecular sieve of the present invention at effective process conditions to produce light olefins.

The term “oxygenate” as used herein designates compounds such as alcohols, ethers and mixtures thereof. Examples of oxygenates include, but are not limited to, methanol and dimethyl ether.

The process of the present invention may be conducted in the presence of one or more diluents which may be present in the oxygenate feed in an amount between about 1 and about 99 molar percent, based on the total number of moles of all feed and diluent components. Diluents include, but are not limited to, helium, argon, nitrogen, carbon monoxide, carbon dioxide, hydrogen, water, paraffins, hydrocarbons (such as methane and the like), aromatic compounds, or mixtures thereof. U.S. Pat. Nos. 4,861,938 and 4,677,242, which are incorporated by reference herein in their entirety, emphasize the use of a diluent to maintain catalyst selectivity toward the production of light olefins, particularly ethylene.

The oxygenate conversion is preferably conducted in the vapor phase such that the oxygenate feedstock is contacted in a vapor phase in a reaction zone with the molecular sieve of this invention at effective process conditions to produce hydrocarbons, i.e., an effective temperature, pressure, weight hourly space velocity (WHSV) and, optionally, an effective amount of diluent. The process is conducted for a period of time sufficient to produce the desired light olefins. In general, the residence time employed to produce the desired product can vary from seconds to a number of hours. It will be readily appreciated that the residence time will be determined to a significant extent by the reaction temperature, the molecular sieve catalyst, the WHSV, the phase (liquid or vapor) and process design characteristics. The oxygenate feedstock flow rate affects olefin production. Increasing the feedstock flow rate increases WHSV and enhances the formation of olefin production relative to paraffin production. However, the enhanced olefin production relative to paraffin production is offset by a diminished conversion of oxygenate to hydrocarbons.

The oxygenate conversion process is effectively carried out over a wide range of pressures, including autogenous pressures. At pressures between about 0.01 atmospheres (0.1 kPa) and about 1000 atmospheres (101.3 kPa), the formation of light olefins will be affected although the optimum amount of product will not necessarily be formed at all pressures. The preferred pressure is between about 0.01 atmospheres (0.1 kPa) and about 100 atmospheres (10.13 kPa). More preferably, the pressure will range from about 1 to about 10 atmospheres (101.3 kPa to 1.013 Mpa). The pressures referred to herein are exclusive of the diluent, if any, that is present and refer to the partial pressure of the feedstock as it relates to oxygenate compounds.

The temperature which may be employed in the oxygenate conversion process may vary over a wide range depending, at least in part, on the molecular sieve catalyst. In general, the process can be conducted at an effective temperature between about 200° C. and about 700° C. At the lower end of the temperature range, and thus generally at a lower rate of reaction, the formation of the desired light olefins may become low. At the upper end of the range, the process may not form an optimum amount of light olefins and catalyst deactivation may be rapid.

The molecular sieve catalyst preferably is incorporated into solid particles in which the catalyst is present in an amount effective to promote the desired conversion of oxygenates to light olefins. In one aspect, the solid particles comprise a catalytically effective amount of the catalyst and at least one matrix material selected from the group consisting of binder materials, filler materials and mixtures thereof to provide a desired property or properties, e.g., desired catalyst dilution, mechanical strength and the like to the solid particles. Such matrix materials are often, to some extent, porous in nature and may or may not be effective to promote the desired reaction. Filler and binder materials include, for example, synthetic and naturally occurring substances such as metal oxides, clays, silicas, aluminas, silica-aluminas, silica-magnesias, silica-zirconias, silica-thorias and the like. If matrix materials are included in the catalyst composition, the molecular sieve preferably comprises about 1 to 99%, more preferably about 5 to 90%, and still more preferably about 10 to 80% by weight of the total composition.

### EXAMPLES

### Examples 1-14

Boron-containing CHA is synthesized by preparing the gel compositions, i.e., reaction mixtures, having the compositions, in terms of mole ratios, shown in the table below. The resulting gel is placed in a Parr bomb reactor and heated in an oven at the temperature indicated below while rotating at the speed indicated below. Products are analyzed by X-ray diffraction (XRD) and found to be boron-containing molecular sieves having the CHA structure. The source of silicon oxide is Cabosil M-5 fumed silica or HiSil 233 amorphous silica (0.208 wt. % alumina). The source of boron oxide is boric acid and the source of aluminum oxide is Reheis F 2000 alumina.

| Ex. # | SiO₂/B₂O₃ | SiO₂/Al₂O₃ | H₂O/SiO₂ | OH—/SiO₂ | Na+/SiO₂ | SDA/SiO₂ | Rx Cond.¹ | Seeds | %1-ada² |
|---|---|---|---|---|---|---|---|---|---|
| 1 | 2.51 | 1,010 | 23.51 | 0.25 | 0.20 | 0.25 | 140/43/5 d | yes | 100 |
| 2 | 12.01 | 1,010 | 22.74 | 0.25 | 0.08 | 0.25 | 140/43/5 d | yes | 100 |
| 3 | 12.33 | 1,010 | 22.51 | 0.25 | 0.08 | 0.25 | 140/43/5 d | yes | 100 |
| 4 | 12.07 | 288,900 | 23.00 | 0.26 | 0.09 | 0.26 | 140/43/5 d | no | 100 |
| 5 | 12.33 | 37,129 | 22.51 | 0.25 | 0.09 | 0.25 | 140/43/5 d | yes | 100 |
| 6 | 12.33 | 248,388 | 22.51 | 0.25 | 0.09 | 0.25 | 140/43/5 d | yes | 100 |
| 7 | 12.33 | 248,388 | 22.53 | 0.25 | 0.09 | 0.25 | 140/43/5 d | yes | 100 |
| 8 | 12.33 | 248,388 | 22.53 | 0.25 | 0.00 | 0.25 | 140/43/5 d | yes | 100 |
| 9 | 12.33 | 248,388 | 22.51 | 0.25 | 0.09 | 0.25 | 160/43/4 d | yes | 100 |
| 10 | 11.99 | 288,900 | 23.18 | 0.26 | 0.09 | 0.26 | 160/43/4 d | no | 100 |
| 11 | 12.13 | 288,900 | 32.22 | 0.43 | 0.21 | 0.21 | 160/43/4 d | no | 100 |
| 12 | 11.99 | 288,900 | 23.16 | 0.26 | 0.00 | 0.26 | 160/43/4 d | no | 100 |
| 13 | 11.99 | 288,900 | 23.18 | 0.26 | 0.09 | 0.26 | 160/43/4 d | no | 100 |
| 14 | 3.08 | 248,388 | 22.51 | 0.25 | 0.00 | 0.25 | 160/43/6 d | yes | 100 |

¹° C./RPM/Days
²1-ada = Quaternary ammonium cation derived from 1-adamantamine

### Examples 15-20

### Deboronation

Boron is removed from samples of the molecular sieves prepared as described in Example 13 above and then calcined. The sample is heated in an acid solution under the conditions indicated in the table below. The results are shown in the table.

| | Ex. No. | | | | | | |
|---|---|---|---|---|---|---|---|
| | Starting | Deboronation Rx | | | | | |
| | (B) SSZ-13 | 15 | 16 | 17 | 18 | 19 | 20 |
| Acid used | — | Acetic acid | acetic acid | acetic acid | HCl | HCl | HCl |
| Acid Molarity | — | 1.0 M | 0.01 M | 0.0001 M | 0.01 M | 0.001 M | 0.0001 M |
| Rx Cond. | — | 45 C./0 rpm/19 hr | 45 C./0 rpm/19 hr | 45 C./0 rpm/19 hr | 45 C./0 rpm/19 hr | 45 C./0 rpm/19 hr | 45 C./0 rpm/19 hr |

Analysis Results

| | Untreated | Treated | Treated | Treated | Treated | Treated | Treated |
|---|---|---|---|---|---|---|---|
| Boron | 0.66% | 614 ppm | 513 ppm | 420 ppm | 421 ppm | 506 ppm | 552 ppm |

What is claimed is:

1. A process for the production of light olefins from a feedstock comprising an oxygenate or mixture of oxygenates, the process comprising reacting the feedstock at effective conditions over a catalyst comprising boron-containing molecular sieve having the CHA crystal structure and comprising (1) silicon oxide and (2) boron oxide or a combination of boron oxide and aluminum oxide, iron oxide, titanium oxide, gallium oxide and mixtures thereof.

2. The process of claim 1 wherein the light olefins are ethylene, propylene, butylene or mixtures thereof.

3. The process of claim 2 wherein the light olefin is ethylene.

4. The process of claim 1 wherein the oxygenate is methanol, dimethyl ether or a mixture thereof.

5. The process of claim 4 wherein the oxygenate is methanol.

6. The process of claim 1 wherein oxide (2) is more than 50% boron oxide on a molar basis.
